(19) United States
(12) Patent Application Publication
(10) Pub. No.: US 2007/0179715 A1
(43) Pub. Date: Aug. 2, 2007

(54) Analyzer, information processing device and computer program product

(75) Inventor: Shunsuke Ariyoshi, Kobe (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION

(21) Appl. No.: 11/518,314

(22) Filed: Sep. 11, 2006

(30) Foreign Application Priority Data
Sep. 14, 2005 (JP) JP 2005-266566

(51) Int. Cl. G06F 19/00; G01N 33/48; C12M 1/00

(52) U.S. Cl. 702/19; 435/283.1

(57) ABSTRACT

An analyzer analyzes a specimen, and generates analysis result. The analysis result includes displaying information used for displaying a graphical analysis result. The analyzer allows to specify a selection criteria of analysis result and select the analysis results by specified selection criteria. The analyzer displays graphical analysis results based on the displaying information included the analysis results selected by the selecting part.

### FIELD OF THE INVENTION

The present invention relates to an analyzer, an information processing device and a computer program product.

### BACKGROUND

In the field of clinical examinations, various types of analyzers are used to perform analyses of components of proteins, cells and the like contained in samples such as blood and urine. Blood analyzers count the numbers of red blood cells, white blood cells, and platelets contained in biological samples of blood, and classify white blood cells in five categories of lymphocytes, monocytes, neutrophils, eosinophils, and basophils. The model SF-3000 blood analyzer (Sysmex Corporation), for example, divides a blood sample into a plurality of aliquots, then adds a first white blood cell classifying reagent to one aliquot to prepare a first sample for white blood cell classification. Thereafter, a flow of the first sample is formed, and the intensity of the low-angle scattered light and the intensity of the high-angle scattered light generated when the flow is irradiated by light are detected using an optical flow cytometer. Then, scattergrams are prepared using the low-angle scattered light intensity and high-angle scattered light intensity, and the white blood cells are classified as lymphocytes, monocytes, granulocyte, and eosinophils. The SF-3000 also prepares a second sample by adding white blood cell classifying reagent to another aliquot of the blood sample, and preparing a similar scattergram based on optical measurement results of the second sample to classify basophils and other white blood cells. Thus, the SF-3000 is capable of classifying five types of white blood cell (refer to U.S. Pat. No. 5,677,183).

Abnormal cells such as blast (juvenile cells) and the like appear in addition to normal white blood cells and red blood cells due to various diseases and pathologies, for example, in such biological samples. In conventional analyzers, the presence of abnormal cells is detected by analyzing biological samples via various methods, and when abnormal cells are detected, a detection message, for example, of the abnormal cell is output (flagging) Furthermore, since there are individual differences between biological samples, differences arise frequency position of abnormal cells and normal cells in the scattergrams of different specimens. Variances also occur among analyzers relative to the analysis results for identical specimens due to differences of environment when the apparatus was manufactured, maintenance condition of the apparatus and the like. A construction method for a determining region to absorb such variances among apparatuses and individual differences among specimens has been disclosed (refer to U.S. Pat. No. 5,735,274).

Blood coagulation analyzers are known as one type of the above mentioned analyzer. A blood coagulation analyzer adds a coagulation reagent to a biological specimen of blood plasma, checks the process of the coagulating plasma via an optical detecting device, and measures the coagulability of the biological sample. In such blood coagulation analyzers, the change in optical information (for example, scattered light intensity) of a sample with time is measured, and the time until the optical information attains a predetermined value is designated as the coagulation time. However, blood coagulation is a reaction that occurs in many stages, and when there is some abnormality in the reaction process, the reaction curve differs from the reaction curve when a normal specimen is measured normally, such that there is concern that an erroneous coagulation time will be output. There are various methods of detecting the generation of such an anomaly by monitoring the reaction curve (refer to US Publication No. 2003/0138962).

The abnormalities arising in apparatuses and specimens are extremely varied, and appearances of abnormalities in analyzers are also quite varied. Even lacking such abnormalities, there will be variations in results for specimens and apparatuses. Therefore, it is difficult to monitor all variations between apparatuses and between specimens and to monitor abnormalities that may occur in specimens and apparatuses even when individual analysis results are monitored by a variety of monitoring means as in conventional analyzers. Scattergrams and coagulation curves include much greater amounts of information compared to the analysis results obtained as numeric values, and although the presence of abnormalities, types of abnormalities, and variations between analyzers and between specimens may be determined by a user visually monitoring the scattergrams and graphs such as coagulation curves, it is difficult to accurately determine the presence of abnormalities, types of abnormalities, and variations between analyzers and between specimens since the relative differences with a normal measurement result can not be grasped by verifying a single scattergram (or graph). Furthermore, while a user might glance at several scattergrams (or graphs), when each scattergram showing different distribution of blood cells are simultaneously displayed as in the case of scattergrams of a plurality of specimens including specimens of various diseases, it is difficult to determine which data are normal and the presence of abnormalities and their types among specimens, and variations among apparatuses and among specimens.

### SUMMARY

The scope of the present invention is defined solely by the appended claims, and is not affected to any degree by the statements within this summary.

The first aspect of the present invention relates to an analyzer for analyzing specimens comprising:

an analyzing part for analysing a specimen and generating an analysis result which includes displaying information used for displaying a graphical analysis result;

a memory for storing the analysis results generated by the analyzing part;

a specifying part for specifying selection criteria of analysis results;

a selecting part for selecting analysis results stored in the memory by the specified selection criteria;

a display part; and

a display controlling part for displaying graphical analysis results on the display part based on the displaying information included the analysis results selected by the selecting part.

The second aspect of the present invention relates to an information processing device comprising:

an obtaining part for obtaining a measurement result of a specimen from a measuring device;

an analyzing part for analysing the obtained measurement result and generating an analysis result which includes displaying information used for displaying a graphical analysis result;

a memory for storing the analysis results generated by the analyzing part;

a specifying part for specifying selection criteria of analysis results;

a selecting part for selecting analysis results stored in the memory by the specified selection criteria;

a display part; and

a display controlling part for displaying graphical analysis results on the display part based on the displaying information included the analysis results selected by the selecting part.

The third aspect of the present invention relates to a computer program product stored in a computer-readable medium which is executed by a computer having a memory and a display, comprising:

a program code for analyzing a specimen and generating an analysis result which includes displaying information used for displaying a graphical analysis result;

a program code for storing the analysis result generated by the analyzing part in the memory;

a program code for specifying selection criteria of analysis results;

a program code for selecting analysis results stored in the memory by the specified selection criteria; and

a program code for displaying graphical analysis results on the display part based on the displaying information included the analysis results selected by the selecting part.

### BRIEF DESCRIPTION OF THE DRAWINGS

FIG. 1 is a screen displaying a scattergram for each specimen in an embodiment of the sample information processing apparatus;

FIG. 2 is a detailed screen of a single specimen;

FIG. 3 is a screen listing numeric data for a plurality of specimens;

FIG. 4 is a screen displaying the particle size distribution for each specimen;

FIG. 5 is a screen displaying a subscreen for the rearranging operation;

FIG. 6 is a screen displaying a subscreen for the filtering operation;

FIG. 7 shows the structure of the sample information processing apparatus;

FIG. 8 shows the structure of the optical system used in flow cytometry;

FIG. 9 is a block diagram of the sample information processing apparatus by function;

FIG. 10 is an example of a scattergram plotted with the side scattered light signals on the horizontal axis and the side fluorescent light signals on the vertical axis;

FIG. 11 is a flow chart of the operation of the sample information processing program;

FIG. 12 is a schematic view illustrating the measurement principle of the biological activity method; and

FIG. 13 is a screen displaying four rows of five coagulation curve graphs for each sample.

### DETAILED DESCRIPTION OF THE EMBODIMENTS

The embodiments of the present invention are described below based on the drawings.

### FIRST EMBODIMENT

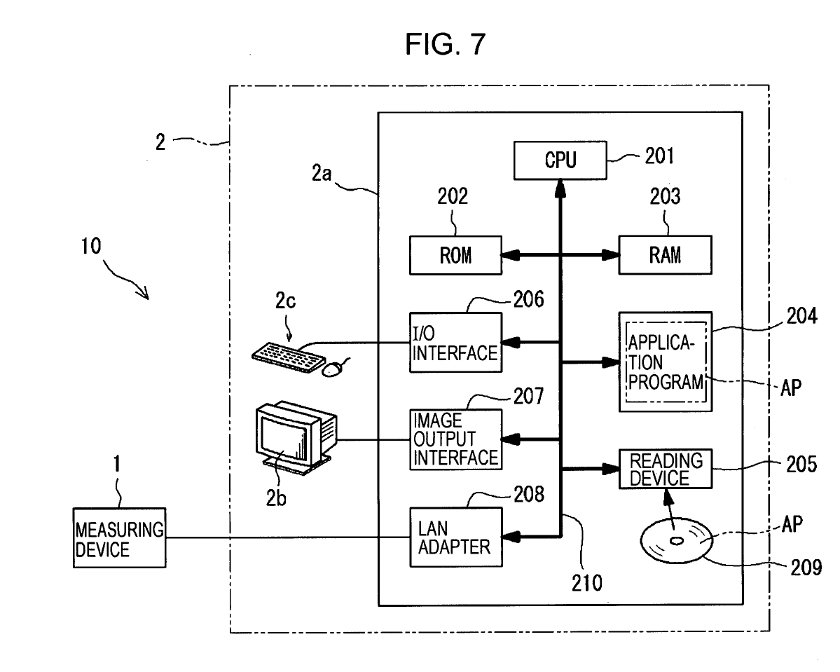

FIG. 7 is a block diagram showing the hardware structure of a multi-item automatic blood analyzer of the first embodiment. In the drawing, an apparatus 10 is configured by a measuring device 1, which functions as the main part for measuring blood cells, and a computer 2 for analysis and processing of the measurement results, both being mutually connected to a LAN.

The computer 2 is mainly configured by a body 2a, display 2b, and input device 2c such as a mouse and keyboard as structural elements. The body 2a is mainly configured by a CPU 201, ROM 202, RAM 203, hard disk 204, reading device 205, input/output (I/O) interface 206, image output interface 207, and LAN adapter 208 as structural elements, which are connected via a bus 210 so as to be capable of data communication.

The CPU 201 executes computer programs stored in the ROM 202 and computer programs loaded in the RAM 203. Then, each function block is realized by the CPU 201 executing an application program AP, which is described later, and the computer 2 functions as the information processing device of the automatic blood analyzer.

The ROM 202 is configured by a mask ROM, PROM, EPROM, EEPROM or the like, and stores computer programs executed by the CPU 201 and data used by those programs.

The RAM 203 is configured by an SRAM or DRAM or the like. The RAM 203 is used when reading the computer programs stored in the ROM 202 and hard disk 204. The RAM 203 is used as a work area for the CPU 201 when the computer programs are executed.

An operating system, application programs, and various computer programs executed by the CPU 201 and data used in the execution of the computer programs are installed on the hard disk 204. The application program AP, which is described later, is also installed on the hard disk 204.

The reading device 205 is configured by a floppy disk drive, CD-ROM drive, DVD-ROM drive or the like, and is capable of reading computer programs or data stored on a portable recording medium 209. The portable recording medium 209 stores the application program AP that allows the computer to function as the information processing device of the analyzer 10; the application program AP of the present invention can be read from the portable recording medium 209 by the CPU 201 and installed on the hard disk 204.

The application program AP need not be provided on the portable recording medium 209 in as much as the application program AP may be provided over an electric communication line from an external device connected to the computer 2 via an electric communication line (wire or wireless) so as t be capable of communication. For example, the application program AP may be installed on the hard disk of a server computer on the internet, such that the server computer can be accessed by the computer 2 which then downloads the computer program and installs the computer program on the hard disk 204.

Furthermore, an operating system capable of providing a graphical user interface, for example, Windows (registered trademark of Microsoft Corporation), is installed on the hard disk 240. In the following description, the application program AP of the present embodiment operates in the environment of this operating system.

The I/O interface 206 is configured by, for example, a serial interface such as a USB, IEEE 1394, RS-232C or the like, parallel interface such as a SCSI, IDE, IEEE 1284 or the like, and analog interface such as a D/A converter, A/D converter or the like. The I/O interface 206 is connected to the input device 2c that includes a keyboard, mouse and the like, and receives data from the body 2a when a user operates the input device 2c.

The image output interface 207 is connected to the display 2b configured by an LCD, CRT or the like, and image signals corresponding to the image data received from the CPU 201 are output to the display 2b. The display 2b displays an image (screen) in accordance with the input image signals. The LAN adapter 208 is connected to the measuring device 1 by an Ethernet communication method.

The measuring device 1 is capable of analyzing blood cells using a semiconductor laser and flow cytometry. The flow cytometric method used forms a thin flow of a sample containing blood cell particles, and irradiating the flow with laser light to obtain optical information. This optical information is particle measurement data of the sample.

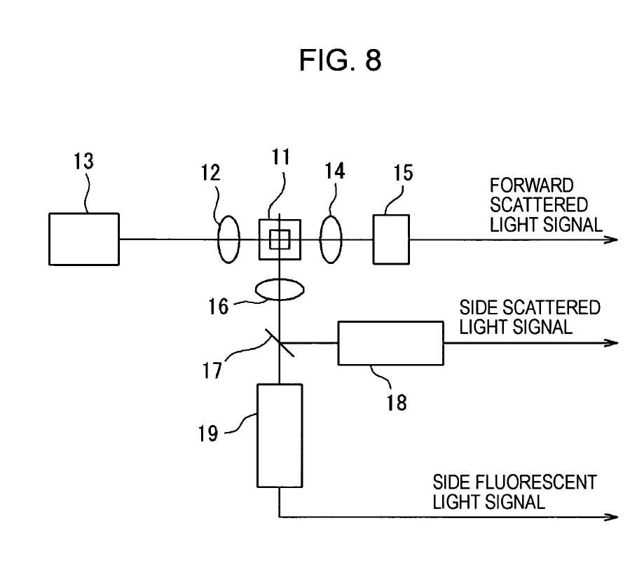

FIG. 8 shows the structure of the optical system used in flow cytometry. In the drawing, a condenser lens 12 focuses the laser light emitted from a light source 13 on a sheath flow cell 11, and a collective lens for collecting the forward scattered light from the particles to a photodiode 15. Another collective lens 16 collects the side scattered light and side fluorescent light from the particles to a dichroic mirror 17. The dichroic mirror 17 reflected the side scattered light to a photomultiplier 18, and the side fluorescent light is transmitted through the dichroic mirror to a photomultiplier 19. The light signals reflect the characteristics of the particles. The photodiode 15, photomultiplier 18 and photomultiplier 19 convert the light signals to electrical signals, and respectively output forward scattered light signal (FSC), side scattered light signal (SSC), and side fluorescent light signal (SFL). These output signals are subjected to amplification, A/D conversion, predetermined signal processing and the like so as to be converted to particle measurement data which are the sent to the computer 2.

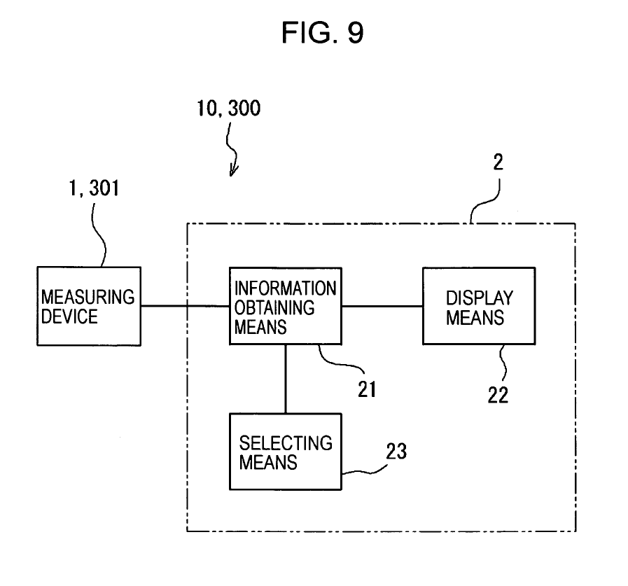

FIG. 9 is a block diagram showing the functions of the computer 2 relating to the present invention. That is, the computer 2 with the installed sample information processing program is provided with an information obtaining means 21 for realizing the function of obtaining image data for displaying the analysis results of a sample, display means 22 for realizing the function of aligning and displaying image data of a plurality of samples on the display 2b, and a selecting means 23 for realizing the function of enabling an information processing operation to be performed on the individual displayed image data selected by the input device 2c.

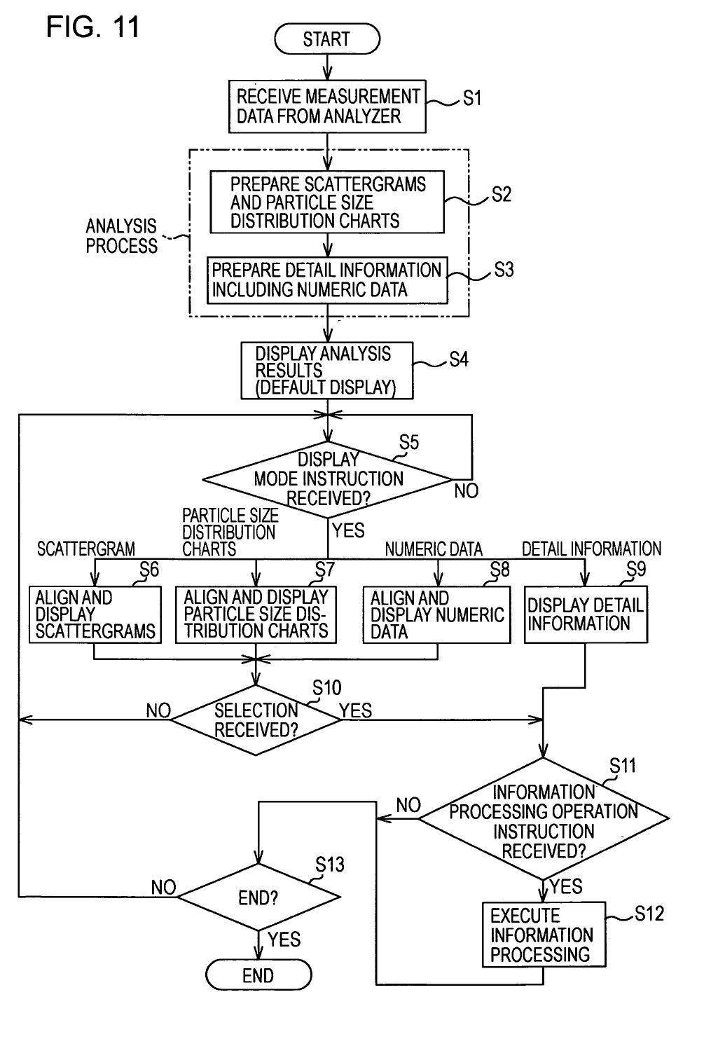

FIG. 11 is a flow chart showing the operation of the sample information processing program for realizing these function on the computer 2. The function of the information obtaining means 21 is mainly realized by steps S1˜S3, the function of the display means 22 is mainly realized by steps S4˜S9, and the functions of the selecting means 23 is mainly realized by steps S10˜S12.

Each step of the flow chart of FIG. 11 is described below. First, in step S1, the computer 2 receives particle measurement data from the measurement device 1. The computer 2 subjects the received particle measurement data to analysis processing, that is, prepares a scattergram and particle size distribution diagram (step S2), and then prepares detail information that includes numeric data. The computer 2 displays the analysis results on the display 2b (step S4). The screen at this time is a default display screen determined beforehand.

The computer 2 awaits the display mode instruction operation (step S5), and performs the indicated display when an instruction is received. The instruction is one of four types calling for scattergrams, particle size distribution charts, numeric data, and detail information, and the computer 2 displays the specified content on the display 2b (steps S6, S7, S8, S9). In the cases of scattergrams, particle size distribution charts, and numeric data, the computer 2 awaits the selection operation (step S10), and once the selection has been received advances to step S11. Furthermore, when displaying the detail information, the process advances directly to step S11.

In step S11, the computer 2 determines whether or not an information processing operation instruction has been received, and when an instruction has been received the information processing is executed (step S12), and thereafter a determination is made as to whether or not the process ends (step S13). When an information processing operation instruction is not received, the determination of whether or not the process ends is made immediately (step S13). When the process has not ended in step S13, the computer 2 returns to step S5 and repeats the above process, and when the process ends in step S13, the processes of the flow chart end.

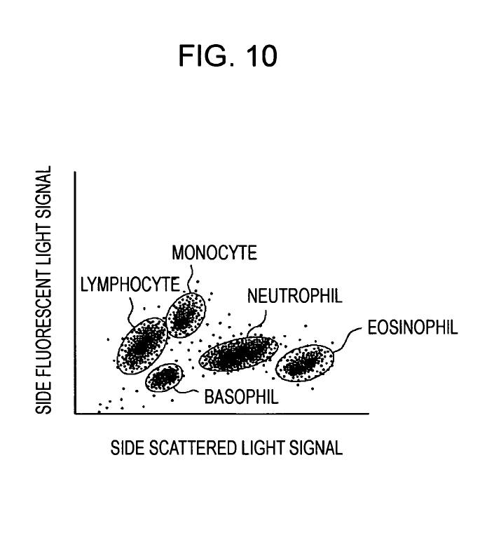

The operation of the sample information processing program is described below in terms of specifics drawn from the operation screens. This program operates in the environment provided by Windows (registered trademark of the Microsoft Corporation). The computer 2 on which this program is installed stores particle measurement obtained from the measurement device 1 for each sample, and creates and displays scattergrams respectively plotting on the vertical and horizontal axes both the forward scattered light component and the side scattered light and side fluorescent light component included in the particle measurement data. The computer 2 measures the white blood cell system that includes white blood cells, basophils and the like, measures the nucleated red blood cells, and measures reticulocytes and platelets by analyzing the scattergrams. FIG. 10 is an example of a scattergram plotted with the side scattered light signals on the horizontal axis and the side fluorescent light signals on the vertical axis, and from this scattergram the white blood cells can be classified in five categories (eosinophils, neutrophils, monocytes, lymphocytes, basophils). The computer 2 prepares image data, and obtains detail information that includes numeric data such as, for example, the number of cells of each type, designates the obtained information as image data and returns.

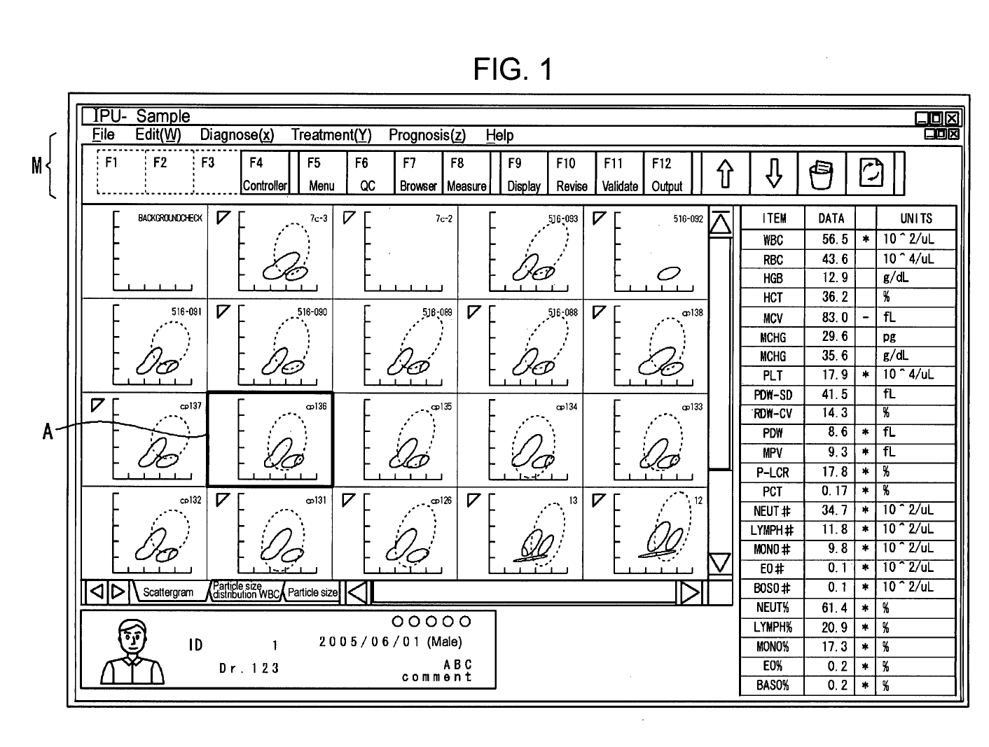

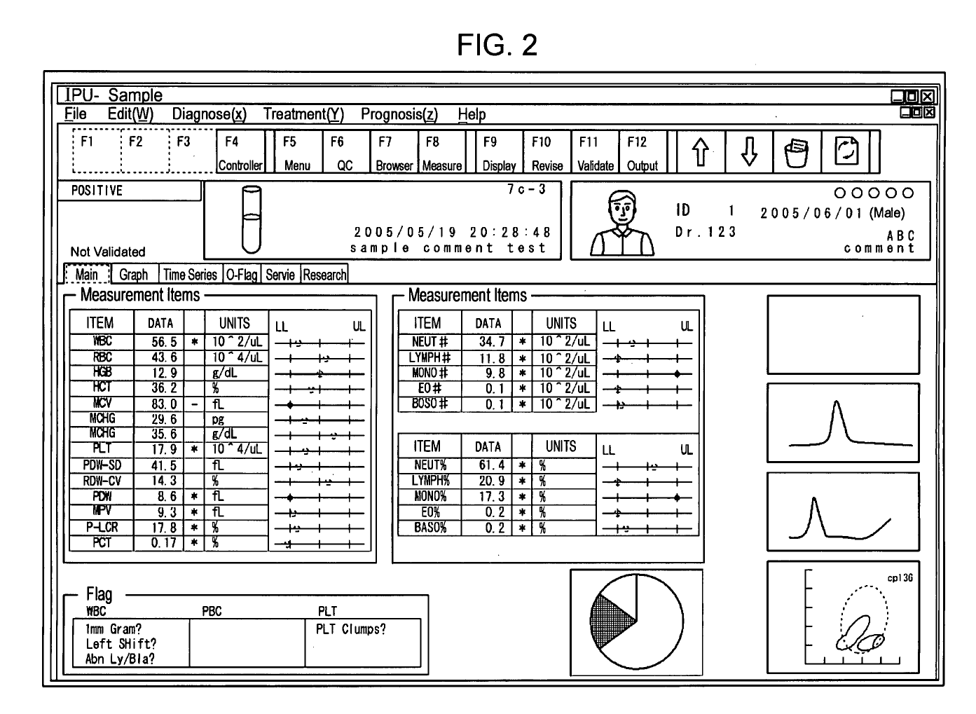

FIG. 1 is a screen that displays four rows of five scattergrams for each sample. This screen is not simply for viewing, it is also an entry screen for receiving the selection operation via the input device 2c. That is, an optional sample A can be selected directly from this screen using the input device 2c (keyboard or mouse or the like). Information regarding the patient who supplied the sample A is displayed at the bottom left of the screen. Various types of operations (information processing operations) are allocated in the menu bar M, which can be performed on the selected sample A. The detail information screen shown in FIG. 2 is displayed by double-clicking on the sample A using the mouse, or selecting the sample A and pressing the enter key. All information obtained for one sample is displayed on this screen. When an operation is performed to close the screen or the escape key is pressed, the screen returns to the original screen shown in FIG. 1.

When the image data (scattergrams) of a plurality of samples is aligned and displayed as shown in FIG. 1, an operating environment is provided that readily allows relative consideration of all samples, such that the detection of anomalies (anomalies of the patient and anomalies of the apparatus) and variance between samples and between apparatuses can be easily detected. For example, when the image data of a plurality of samples are aligned and displayed such that one image appears markedly different from the others, that sample can be confirmed to be anomalous at a glance, and when a plurality of image data are aligned and arranged in time-series order such that the condition of images change with time, an anomaly occurring in the analyzer can be readily confirmed. Although numeric data are included in part of the information displayed in image data (or particle measurement data) expressed numerically, all the information included in the image data is not included in the numeric data. Conversely, information that can not be displayed as numeric data is hidden in the image data. When image data are displayed in summary so as to allow easy comparison of each sample, an operating environment is provided that is suited for discovering such information. Since the information processing operation can be performed directly from the screen displaying the image data, detail information that includes numeric data can be readily accessed by selecting a sample via the input device 2c when some anomaly or other has been discovered in the image data. Thus, an operating environment is provided in which the measurement results of a sample can be easily considered.

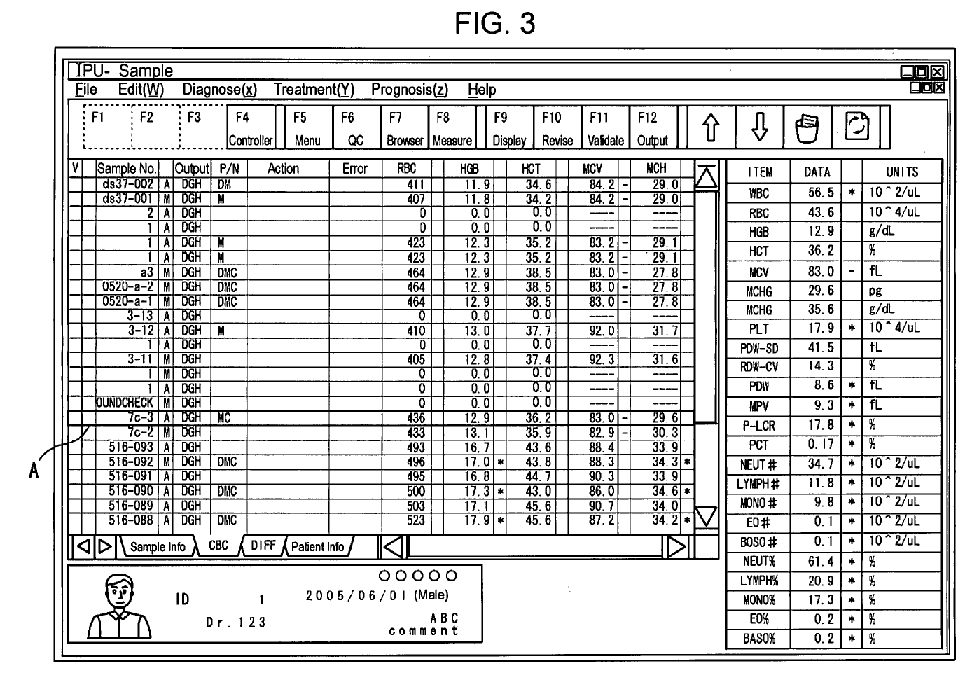

The screen shown in FIG. 3 can be displayed by clicking the display change button on the menu bar from the screen shown in FIG. 1, or by pressing a predetermined function key. In this screen, a list of numeric data is displayed for a plurality of samples, and the numeric data of a selected sample A are displayed similar to FIG. 1. The screen can be restored to the screen of FIG. 1 by the same operation.

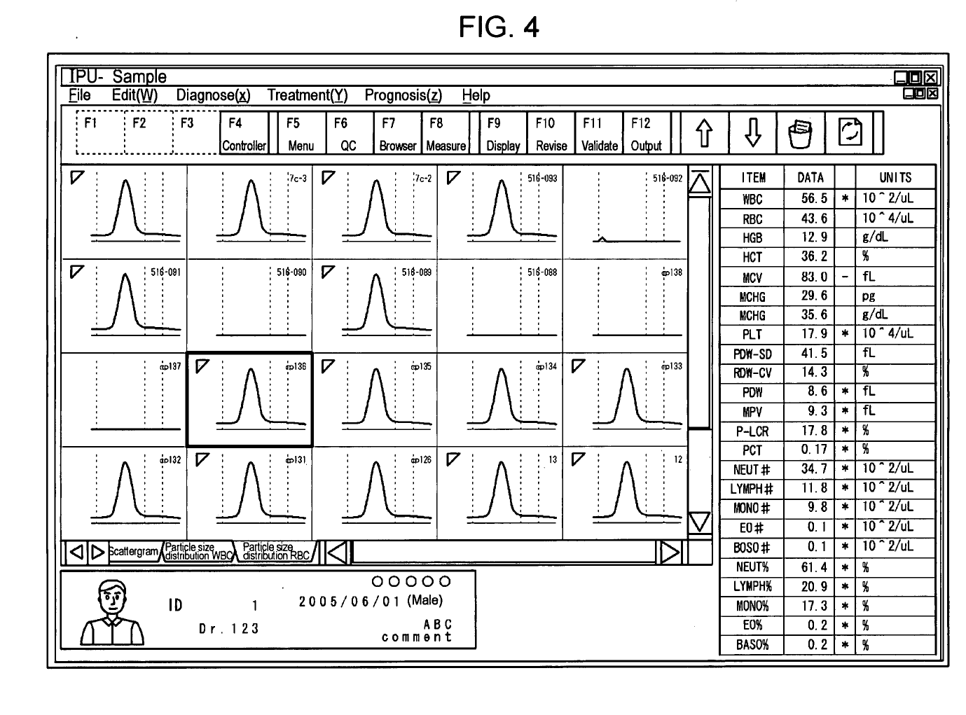

FIG. 4 is a screen displayed by clicking a tab from the screen in FIG. 1, or pressing a tab key. In this case the screen displays particle size distribution charts for each sample rather than the scattergrams of FIG. 1. In this case a sample can also be selected using the input device 2c. Similar to the case of the scattergrams, an operating environment is provided which allows the particle size distributions of each sample to be compared since the distribution charts are displayed on the screen.

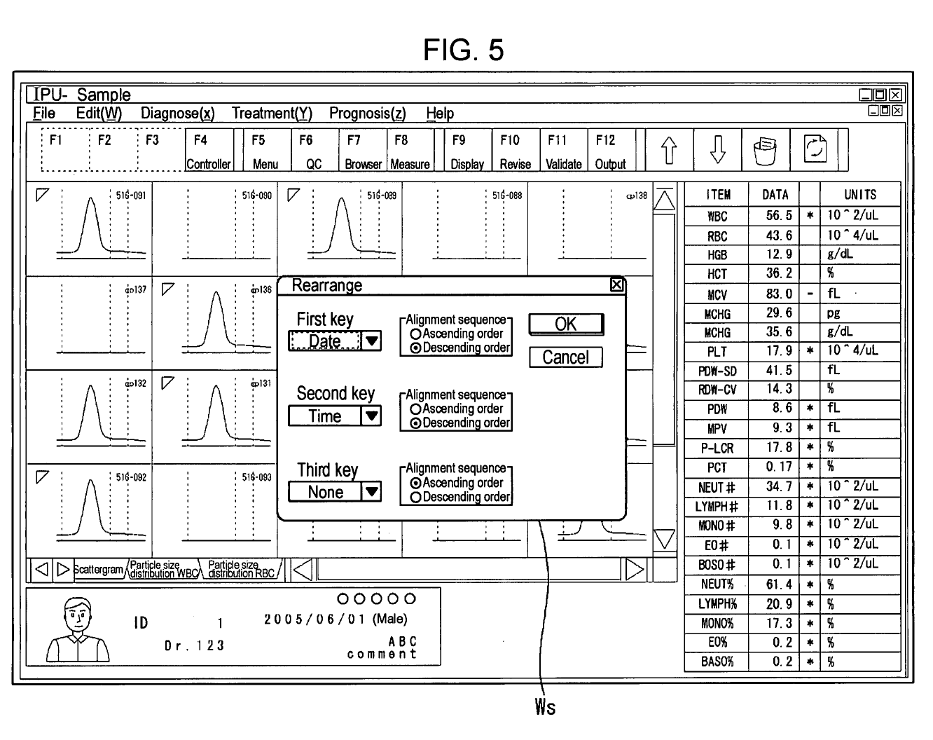

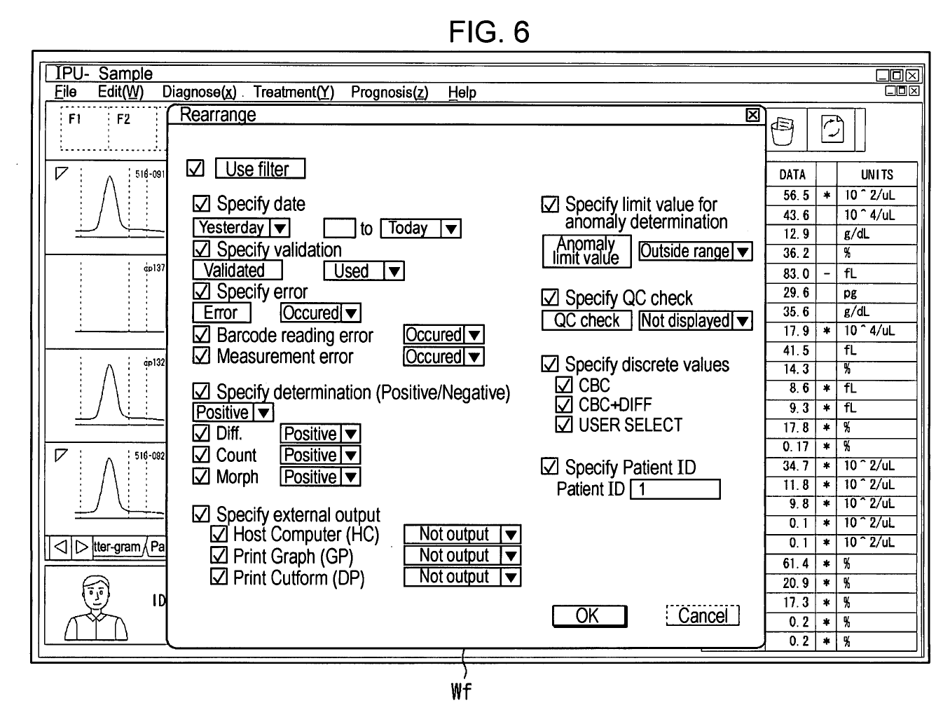

FIG. 5 shows a sub screen Ws that is overlaid on the original screen (in this example the screen shown in FIG. 4) when the “rearrange” operation is performed from the menu bar of any of the screens in FIGS. 1˜4. Rearrangement can be performed for predetermined items such as date and time. Similarly, FIG. 6 shows a sub screen Ws overlaid on the original screen for performing a “filter (extraction” operation via the menu bar on any of the screen of FIGS. 1˜4. The filtering can be performed for any item, such as date, whether or not validation has been performed, presence of error and the like. The filtering items includes analysis conditions such as validation and error. Rearrangement and extraction make sit easier to compare each of the samples.

In the image data display, the image data of the scattergrams and particle size distribution charts may be displayed for a plurality of samples respectively obtained from different patients, and a plurality of samples from the same patient may be aligned and displayed side by side. In the case of different patients, is possible to easily detect anomalies by comparing the samples as previously described. Furthermore, anomalies of the particle measurement data, that is, anomalies of the measuring device 1 (for example, reduced sensitivity, clogging) can easily be detected from the characteristics of the image data together with the samples.

Changes can also be comprehended for samples from the same patient via aligning data in time series. Furthermore, when the measuring device 1 is contaminated, the efficacy of cleaning can be confirmed by changes in the image data if the pre cleaning samples and post cleaning samples are aligned and displayed side by side.

Although the sample information processing apparatus above has been described by way of the example of a blood analyzer, the structure of aligning and displaying image data relating to a plurality of samples and providing a screen from which these samples can be selected is also applicable to analyzers other than blood analyzers, for example, particle analyzers capable of analyzing particles other than blood cells, immunoassay apparatuses for determining the concentration of antigens or antibodies of cancer markers, blood coagulation measuring apparatuses for examining coagulation function of serum and plasma cylinders, biochemical analyzers for measuring enzyme activity which indicates organ function and serum total protein, and urine sedimentation analyzers for quantifying red blood cells, white blood cells, epithelial cells, columnar cells, and microbes in urine.

Furthermore, and various charts in addition to scattergrams, particle size distributions and the like may be used as image data for display on the sample information processing apparatus. For example, radar charts prepared for a plurality of measurement items are applicable to comparisons of a plurality of samples.

### SECOND EMBODIMENT

A second embodiment of the present invention is described below. The analyzer 300 of the second embodiment is a blood coagulation analyzer for analyzing blood coagulation function. The blood coagulation analyzer 300 is provided with a measuring device 301 and computer 2 for analysis processing (refer to FIG. 9). The structure of the computer 2 is identical to that of the first embodiment, with like parts designated by like reference numbers and, therefore, further description is omitted.

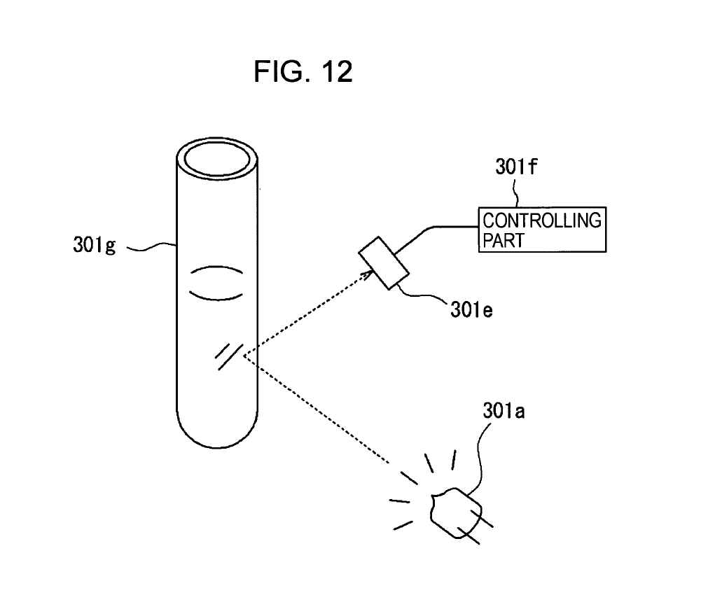

The measuring device 301 has a light-emitting diode 301a and photodiode 301e (refer to FIG. 12), and a heater that is not shown in the drawing. The measuring device 301 measures optical information of the blood using a biological activity method, and sends the measurement data to the computer 2.

FIG. 12 is a schematic view illustrating the measurement principle of the biological activity method. The blood coagulation analyzer 300 has the measurement items of prothrombin time (PT), activated partial thromboplastin time (APTT), and fibrinogen (Fbg); the blood coagulation analyzer 300 divides the blood sample into sufficient aliquots for each measurement item, adds the special reagents used for each measurement item to the aliquots, executes scattered high intensity measurements of each aliquot via the measurement device 301 as described below, and the computer 2 calculates the blood coagulation time based on the measurement results.

As shows in FIG. 12, the light-emitting diode 301a is disposed so as to emit light toward a cuvette 301g that contains sample in the measuring device 301. a photodiode 301e is disposed with the photoreceptive surface facing the cuvette 301g at the side of the cuvette 301g, and the photoreceptive optical axis direction of the photodiode 301e is at approximately 90 degrees to the horizontal relative to the light-emitting optical axis of the light-emitting diode 301a. The light-emitting diode 301a emits light that has an approximate wavelength of 660 nm. A measured quantity of plasma is contained in the cuvette 301g, and coagulation reagent is added after the plasma has been heated for a fixed time. Thereafter, light is irradiated from the light-emitting diode 301a toward the sample, and the scattered light from the sample is received by the photodiode 301e. The amount of received light represents the opacity of the sample, and the sample provides weak scattered light (low opacity) immediately after the reagent has been added such that there is scant change in the amount of received light. However, fibrin clots begin to form in the sample as the reaction progresses, and the sample becomes more opaque in conjunction therewith, such that there is a rapid increase in the scattered light. When the clotting reaction ends, the increase in the scattered light ceases, and the photoreception level becomes stable. The photodiode 301e outputs an electrical signal that corresponds to the amount of received light, and these electrical signals are sent to a controlling part 301f for controlling the operations of each structural element of the measuring device 301, the controlling part 301f being configured by a CPU, ROM, RAM and the like. The data representing the amount of received light are sent to the computer 2, which has executed the sample information processing program, and the computer 2 determines a coagulation curve from the photoreception data, and calculates the coagulation time of the sample thereby. In the computer 2, the concentration or the percentage of activity of the specific blood components can be calculated from the coagulation time.

The computer 2 stores the measurement data provided from the measuring device 301 for each sample, and prepares and displays coagulation curve graphs plotted with the time on the horizontal axis and scattered light intensity on the vertical axis based on the measurement data. The computer 2 calculates prothrombin time (PT), activated partial thromboplastin time (APTT), and coagulation time for fibrinogen (Fbg). Specifically, the computer 2 designates the end of the coagulation reaction at the point at which there is no longer any change in the scattered light intensity obtained via the measurement data, and calculates the elapsed time from the start of the reaction until one half of the scattered light intensity at the end of the coagulation reaction has been attained as the coagulation time. The computer 2 associates and stores the prepared coagulation curves and numeric data such as coagulation time and the like.

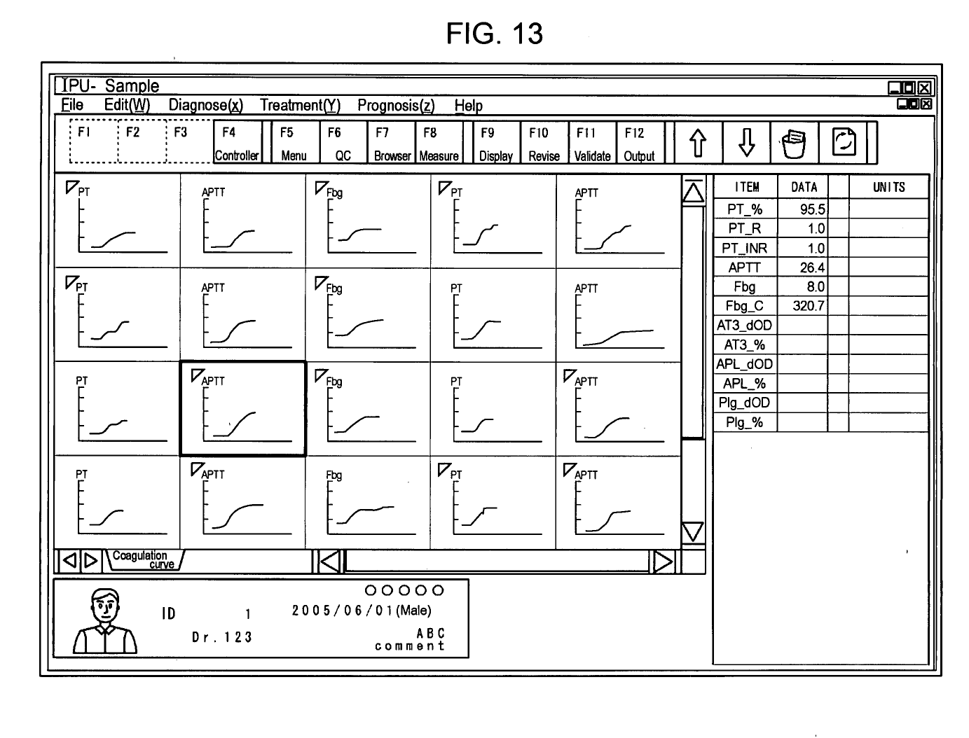

FIG. 13 is a screen displaying four rows of five coagulation curve graphs for each sample. This screen has the same screen layout as the scattergram summary display described via FIG. 1 in the first embodiment. That is, coagulation curve graphs rather than scattergrams are summarized and displayed in the screen shown in FIG. 13, and numeric data are displayed on the right side when a selected coagulation curve graph is clicked. Similar to the first embodiment, a user sets the various selection criteria to select the samples for which the coagulation curve graphs will be displayed. In other respects the operation of the computer 2 in this screen is identical to the operation of the computer 2 in the screen shown in FIG. 1 of the first embodiment and, therefore, further description is omitted.

According to this configuration, since a plurality of coagulation curve graphs are aligned and displayed, a user can compare the various coagulation curve graphs on a single screen, and easily and accurately determine the occurrence of an anomaly, type of anomaly, and variances between samples and measuring devices. Since scattergrams and coagulation curve graphs relating to each sample selected by optional selection criteria are aligned and displayed in the first and second embodiments, a user can easily determine the occurrence of an anomaly, type of anomaly, and variances between samples and measuring devices by visually confirming the scattergram and graph by selecting only the sample that can be used to distinguish the occurrence of an anomaly, type of anomaly, and variances between samples and measuring devices.

Furthermore, in the first and second embodiments, the computer 2 stores the scattergrams and coagulation curve graphs as image data, which can be read and displayed when displaying summaries. Thus, when the computer 2 displays summaries of the scattergrams and coagulation curve graphs, the screen display can be rapidly executed by simply reading and displaying the image data. The present invention is not limited to this configuration inasmuch as data such as the coordinate values required to prepare the scattergrams and graphs may be stored beforehand, such that when the scattergrams and graphs are to be displayed, the coordinate value data are read to prepare the scattergrams or graphs. Therefore, since the scattergrams or graphs are prepared when they are displayed, they can not only be displayed as simple scattergrams or graphs, these scattergrams or graphs may be edited and the display modes may be altered. The scattergrams or graphs need not be stored as image data and may be stored in a data format that allows scattergrams or graphs to be prepared, which is advantageous from the perspective of the quantity of data. The data may also be used in other analysis results and not just to prepare scattergrams or graphs.

As previously mentioned, since displayed scattergrams or graphs are selectable and the information processing operation can be executed for the analysis results of selected samples, the operating characteristics are improved for the user since the information processing operation is performed without switching the display, for example, to a mode for displaying numeric data of a plurality of analysis results. Furthermore, since the scattergrams or graphs provide abundantly more information than mere numeric data, usability is also improved on those frequent occasions when a user needs to visually confirm a plurality of scattergrams or graphs in order to determine the analysis results of the object to be subjected to the information processing operation.

Since numeric data of the analysis results corresponding to selected scattergrams or graphs can be displayed in the screen displaying the scattergrams or graphs, the operational characteristics for the user are improved because numeric data relating to specific samples can be verified without switching from the display of the aligned scattergrams or graphs to a display of numeric data. Moreover, both the numeric data of selected samples and a plurality of scattergrams can be advantageously verified simultaneously.

Since the criteria can be set for selecting analysis results relating to samples collected from the same patient, and since scattergrams or graphs for a plurality of samples of the same patient can be aligned and displayed, any changes in the pathological condition of a given patient can he easily confirmed, for example, when that patient has a particular disease. Although scattergrams or graphs prepared from samples of the same patient typically exhibit similar conditions, when a trend of specific differences appear in the scattergrams or graphs, it may be an indication of variances between measuring devices or change in the condition of a measuring device with time. Therefore, a user or support personnel or the like can verify any variance between devices or change with time in the condition of a device.

Certain advantages accrue since scattergrams or graphs can be aligned and displayed in a predetermined sequence (for example, in time series, or patient series), for example, various analysis results can be investigated such as a user can confirm changes in a pathological condition with time or changes in the condition of a device with time, and can confirm variation in data between patients.

The foregoing detailed description and accompanying drawings have been provided by way of explanation and illustration, and are not intended to limit the scope of the appended claims. Many variations in the presently preferred embodiments illustrated herein will be obvious to one of ordinary skill in the art, and remain within the scope of the appended claims and their equivalents.

What is claimed is:

1. An analyzer for analyzing specimens comprising:
an analyzing part for analysing a specimen and generating an analysis result which includes displaying information used for displaying a graphical analysis result;
a memory for storing the analysis results generated by the analyzing part;
a specifying part for specifying selection criteria of analysis results;
a selecting part for selecting analysis results stored in the memory by the specified selection criteria;
a display part; and
a display controlling part for displaying graphical analysis results on the display part based on the displaying information included the analysis results selected by the selecting part.

2. The analyzer according to claim 1, wherein
the analyzing part comprises:
a measuring part for performing predetermined measurements of a specimen and obtaining measuring data; and
a preparing part for preparing the displaying information of the specimen based on the measurement data obtained by the measuring part,
wherein the displaying information is the graphical analysis result.

3. The analyzer according to claim 1, wherein
the display controlling part comprises a preparing part for preparing graphical analysis result based on the displaying information, and is configured so as to display the graphical analysis result prepared by the preparing part on the display part.

4. The analyzer according to claim 1, wherein
the graphical analysis result is a scattergram or a graph.

5. The analyzer according to claim 4, wherein
the specimen contains particles, and the scattergram or graph represents particle size distribution.

6. The analyzer according to claim 1, further comprising:
a selection receiving part for receiving selection of at least one of graphic analysis result displayed on the display part;
an instruction receiving part for receiving instruction to process the analysis result related to the selected graphic analysis result; and
a process executing part for executing the process which is instructed.

7. The analyzer according to claim 6, wherein
the analysis result further includes numeric analysis result, and
the display controlling part is configured so as to display on the display part a plurality of graphic analysis results related to a plurality of specimens and numeric analysis result related to the selected graphic analysis result when selection of graphic analysis result has been received by the selection receiving part.

8. The analyzer according to claim 1, wherein
the specifying part is configured so as to specify at least selection criteria for selecting analysis results related to specimens collected from same person.

9. The analyzer according to claim 1, wherein
the display controlling part is configured so as to display the graphical analysis results aligned in a predetermined order on the display part.

10. An information processing device comprising:
an obtaining part for obtaining a measurement result of a specimen from a measuring device;
an analyzing part for analysing the obtained measurement result and generating an analysis result which includes displaying information used for displaying a graphical analysis result;
a memory for storing the analysis results generated by the analyzing part;
a specifying part for specifying selection criteria of analysis results;
a selecting part for selecting analysis results stored in the memory by the specified selection criteria;
a display part; and
a display controlling part for displaying graphical analysis results on the display part based on the displaying information included the analysis results selected by the selecting part.

11. The information processing device according to claim 10, wherein
the analyzing part comprises a preparing part for preparing the displaying information of the specimen based on the obtained measurement data,
wherein the displaying information is the graphical analysis result.

12. The information processing device according to claim 10, wherein
the display controlling part comprises a preparing part for preparing graphical analysis result based on the displaying information, and is configured so as to display the graphical analysis result prepared by the preparing part on the display part.

13. The information processing device according to claim 10, wherein
the graphical analysis result is a scattergram or a graph.

14. The information processing device according to claim 13, wherein
the specimen contains particles, and the scattergram or graph represents particle size distribution.

15. The information processing device according to claim 10, further comprising:
a selection receiving part for receiving selection of at least one of graphic analysis result displayed on the display part;
an instruction receiving part for receiving instruction to process the analysis result related to the selected graphic analysis result; and
a process executing part for executing the process which is instructed.

16. The information processing device according to claim 15, wherein
the analysis result further includes numeric analysis result, and
the display controlling part is configured so as to display on the display part a plurality of graphic analysis results related to a plurality of specimens and numeric analysis result related to the selected graphic analysis result when selection of graphic analysis result has been received by the selection receiving part.

17. The information processing device according to claim 10, wherein
the specifying part is configured so as to specify at least selection criteria for selecting analysis results related to specimens collected from same person.

18. The information processing device according to claim 10, wherein
the display controlling part is configured so as to display the graphical analysis results aligned in a predetermined order on the display part.

19. A computer program product stored in a computer-readable medium which is executed by a computer having a memory and a display, comprising:
a program code for analyzing a specimen and generating an analysis result which includes displaying information used for displaying a graphical analysis result;
a program code for storing the analysis result generated by the analyzing part in the memory;
a program code for specifying selection criteria of analysis results;
a program code for selecting analysis results stored in the memory by the specified selection criteria; and
a program code for displaying graphical analysis results on the display part based on the displaying information included the analysis results selected by the selecting part.

20. The computer program product according to claim 19, wherein
the graphical analysis result is a scattergram or a graph.
